(19) United States
(12) Patent Application Publication
(10) Pub. No.: US 2006/0089635 A1
(43) Pub. Date: Apr. 27, 2006

(54) Methods and apparatus for focused bipolar tissue ablation using an insulated shaft

(75) Inventors: Kimbolt Young, Newtonville, MA (US); Steve M. Anderson, Worcester, MA (US); Paul DiCarlo, Middleboro, MA (US); Jeffrey W. Zerfas, Bloomington, IN (US)

Correspondence Address: Bingham McCuthen, LLP, Suite 1800, Three Embarcadero, San Francisco, CA, 94111-4067, US

(73) Assignee: Scimed Life Systems, Inc., Maple Grove, MN (US)

(21) Appl. No.: 10/971,373

(22) Filed: Oct. 22, 2004

(51) Int. Cl. A61B 18/14

(52) U.S. Cl. 606/41

(57) ABSTRACT

A tissue ablation probe is provided. The probe comprises a proximal electrode element, which includes a proximal electrode stem and a deployable proximal electrode array, and a distal electrode element, which includes a distal electrode stem and a deployable distal electrode array. The probe is configured, such that a majority of electrical energy conveyed between the proximal and distal electrode elements is conveyed between distal termini of the electrode arrays, whereas a relatively small amount of the electrical energy is conveyed between the electrode stems. One or more of the electrodes on the arrays may be optionally insulated to further enhance the electrical characteristics of the arrays.

### FIELD OF THE INVENTION

The invention relates generally to the structure and use of radiofrequency electrosurgical apparatus for the treatment of tissue. More particularly, the invention relates to an electrosurgical system having pairs of electrode arrays, which are deployed to treat large volumes of tissue, particularly for the treatment of tumors in the liver and other tissues and organs.

### BACKGROUND

The delivery of radiofrequency energy to treatment regions within tissue is known for a variety of purposes. Of particular interest to the invention, radiofrequency energy may be delivered to diseased regions in target tissue for the purpose of causing tissue necrosis. For example, the liver is a common depository for metastases of many primary cancers, such as cancers of the stomach, bowel, pancreas, kidney, and lung. Electrosurgical probes for deploying multiple electrodes have been designed for the treatment and necrosis of tumors in the liver and other solid tissues. See, for example, the LeVeen™ Needle Electrode available from Boston Scientific Corporation, which is constructed generally in accordance with published PCT application WO 98/52480.

The probes described in WO 98/52480 comprise a number of independent wire electrodes, which are extended into tissue from the distal end of a cannula. The wire electrodes may then be energized in a monopolar or bipolar fashion to heat and necrose tissue within a defined generally spherical volumetric region of target tissue. In order to assure that the target tissue is adequately treated and to limit damage to adjacent healthy tissues, it is desirable that the array formed by the wire electrodes within the tissue be precisely and uniformly defined.

Despite the significant success that has accompanied use of the LeVeen™ Needle Electrode in treating solid tissue tumors, the ability to treat particular types of tumors has been somewhat limited. For example, the ability to produce very large tissue lesions, for example lesions having volumes greater than 30-35 cm³, has been problematic. In addition, such larger tumors tend to be less spheroidal in shape than smaller tumors. Since the LeVeen™ Needle Electrode produces generally spheroidal lesions, the ability to treat larger, non-spheroidal tumors can be limited. Additionally, the ability to treat highly vascularized tissues and/or tissue near a large blood vessel has also been limited. In the latter cases, heat being introduced by the electrode can be rapidly carried away by circulating blood, making uniform heating and control of temperature in the vascularized tissues difficult. Uniform heating and temperature control of the tissue being treated is, of course, one prerequisite to obtaining homogenous lesions in and around the tumors.

The ability to provide uniform heating and the creation of homogenous tissue lesions is particularly difficult with bipolar devices. The two bipolar electrodes may be placed in regions with quite different perfusion characteristics, and the heating around each pole can be quite different. That is, one pole may be located adjacent to a large blood vessel, while the other pole may be located adjacent to tissue, which is less perfused. Thus, the pole located in the less perfused tissue will heat the tissue immediately surrounding the electrode much more rapidly than the tissue surrounding the opposite polar electrode is heated. In such circumstances, the tissue surrounding one pole may be preferentially heated and necrosed, while the tissue surrounding the other pole will neither be heated nor necrosed sufficiently.

To address these issues, a bipolar dual electrode array probe, as described in U.S. patent application Ser. No. 09/663,048, entitled “Methods and Systems for Focused Bipolar Tissue Ablation,” which is expressly incorporated herein by reference, has been developed. As shown in FIG. 1, one embodiment of a bipolar dual electrode array probe 1 may comprise an elongated shaft 2 from which distal and proximal electrode arrays 3 and 4 may be deployed. These electrode arrays 3 and 4 may be operated in a bipolar mode, such that electrical energy is transmitted between the distal electrode array 3 and proximal electrode array 4 via electrical energy paths (shown as arrows) in order to ablate tissue therebetween. The shaft 2 is electrically conductive and comprises a non-conductive gap 5 in order to electrically isolate the arrays 3 and 4 from each other. As can be seen from FIG. 1, the electrical energy paths follow the path of least resistance through the electrically conductive shaft 2. In particular, electrical energy is transmitted from the distal portion of the shaft 2 adjacent the distal electrode array 3 to the tips of the proximal electrode array 4, and electrical energy is transmitted from the tips of the distal electrode array 3 to the proximal portion of the shaft 2 adjacent the proximal electrode array 4. It has been discovered that, as a result, tissue located radially outward from the center region of the shaft 2 may not be ablated, resulting in an hour-glass shaped ablation region, as illustrated in FIG. 2.

For this reason, it would be desirable to provide improved bipolar electrosurgical methods and systems for more uniformly ablating tumors in the liver and other body organs.

### SUMMARY OF THE INVENTION

In accordance with preferred embodiments of the present inventions, a tissue ablation probe is provided. The probe comprises proximal and distal electrode arrays, each of which has a retracted configuration and a deployed configuration. The probe further comprises a shaft for carrying the electrode arrays. In one embodiment, the electrode arrays are electrically isolated from each other and have respective concave faces that oppose each other when in the deployed configuration, thereby enhancing the bipolar nature of the probe. In forming a concave face, an electrode array may comprise a plurality of individual electrodes that initially move axially and then evert as they are deployed. In another embodiment, the shaft comprises a proximal conductive tube from which the proximal electrode array is deployed, and a distal conductive tube from which the distal electrode array is deployed. The conductive tubes may, e.g., be coaxial relative to each other or may be in a side-by-side relationship.

In accordance with one aspect of the present inventions, the electrode arrays have distal termini, and the shaft has an electrically insulative portion that separates the electrode arrays. In one embodiment, the insulative portion is continuous, but may also have gaps. The shaft portion can be insulated in any one of a variety of ways, but in one embodiment, the insulative shaft portion comprises an electrically conductive wall on which electrically insulative material is disposed. The electrode arrays are separated from each other by a first length when deployed, and the insulative shaft portion spans a second length greater than seventy-five percent of the first length. By way of non-limiting example, the insulative shaft portion may allow most of the electrical current to flow between the electrode arrays, rather than along the normally conductive shaft, thereby enhancing the shape of the resulting tissue ablation. The second length may be further increased relative to the first length (e.g., equal to or greater than) to allow even more electrical current to flow between the electrode arrays.

In accordance with another separate aspect of the present inventions, the shaft has an intervening portion between the electrode arrays. The intervening portion has an electrically conductive proximal region, an electrically conductive distal region, and a non-conductive gap therebetween. The probe further comprises an electrically insulative material covering at least portions of the proximal and distal shaft regions. Optionally, the insulative material may also cover the non-conductive gap. By way of non-limiting example, the application of the insulative material on the conductive shaft provides a convenient means of modifying the amount of current that flows between the electrode arrays. In one embodiment, the insulative material is closer to one of the electrode arrays than the other. In this manner, the flow of electrical current adjacent one array can be modified relative to the other array.

In accordance with still another separate aspect of the present inventions, the probe comprises proximal and distal electrically conductive tubes that are electrically isolated from each other. The proximal electrode array is proximally deployable from and electrically coupled to the proximal tube, and the distal electrode array distally deployable from and electrically coupled to the distal tube. The probe further comprises an electrically insulative material covering at least portions of the proximal and distal tubes. In one embodiment, the insulative material continuously extends from the proximal tube to the distal tube, and, depending on the desired proportion of electrical current conveyed between the electrode arrays, may cover the entirety of the proximal and distal tubes.

In accordance with other preferred embodiments of the present inventions, another tissue ablation probe is provided. The probe comprises a proximal electrode element that includes a proximal electrode stem and a deployable proximal electrode array, which has distal termini and is electrically coupled to a proximal end of the proximal electrode stem when deployed. The probe further comprises a distal electrode element including a distal electrode stem and a deployable distal electrode array, which has distal termini and is electrically coupled to a distal end of the distal electrode stem when deployed. The electrode arrays may have the same features as the electrode array previously described above. The probe is configured, such that a majority of electrical energy conveyed between the proximal and distal electrode elements is conveyed between distal termini of the electrode arrays. In some embodiments, substantially all of the electrical energy conveyed between the proximal and distal electrode elements is conveyed between distal termini of the electrode arrays. In one embodiment, the proximal electrode stem comprises a proximal conductive tube from which the proximal electrode array is deployed, and the distal electrode stem comprises a distal conductive tube from which the distal electrode array is deployed.

In accordance with a preferred method of the present invention, a target tissue region (e.g., a tumor within an organ such as the liver, lung, kidney, pancreas, stomach, uterus, or spleen) is treated. The method comprises deploying a first electrode array on one side of the tissue region, and deploying a second electrode array on another side of the tissue region, such that the electrode arrays define a periphery therebetween. The method further comprises transmitting electrical energy (e.g., at a frequency in the range from 300 kHz to 1.2 MHz and at a power in the range of 50 W to 300 W) from the first electrode array to the second electrode array, so that the tissue region along the periphery is initially ablated. In one method, the core of the tissue region within the periphery is subsequently ablated. By way of non-limiting example, this method will typically produce a more uniform ablation.

In accordance with another preferred embodiment of the present inventions, a tissue ablation probe is provided. The probe comprises an array of needle electrodes having a retracted configuration and a deployed configuration, and a shaft carrying the electrode array. The probe further comprises an electrically insulative material partially disposed on at least one needle electrode of each array, whereby a tip of the needle electrode(s) is left exposed. For example, the insulative material can be disposed on the needle electrode(s) at a point on the shaft from which the needle electrodes deploy to somewhere along the length of the needle electrode(s). In one embodiment, the insulative material may be partially disposed on all the needle electrodes of each array, whereby tips of the needle electrodes are left exposed. By way of non-limiting example, the electrical insulation of portions of the needle electrode(s) allows the electrical current to be more focused at the tips of the electrode array, thereby providing for a greater tissue ablation. In one embodiment, the probe comprises proximal and distal electrode arrays on which the electrically insulative material is applied. In this case, the shaft and electrode arrays can optionally have the same features as the electrode arrays previously described above to further enhance bipolar ablation between the arrays.

Other and further aspects and features of the invention will be evident from reading the following detailed description of the preferred embodiments, which are intended to illustrate, not limit, the invention.

### BRIEF DESCRIPTION OF DRAWINGS

The drawings illustrate the design and utility of preferred embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a plan view of a prior art bipolar probe;

FIG. 2 is a plan view of an ablation lesion resulting from the probe of FIG. 1;

FIG. 3 is schematic illustration of one set of bipolar electrode arrays arranged in accordance with the present invention;

FIG. 4 is schematic illustration of another set of bipolar electrode arrays arranged in accordance with the present invention;

FIG. 5 is schematic illustration of still another set of bipolar electrode arrays arranged in accordance with the present invention;

FIGS. 6A-6B are schematic illustrations of the progression of tissue ablation achieved with the bipolar electrode arrays of FIG. 3 are electrically activated;

FIGS. 7A-7B are schematic illustrations of the progression of tissue ablation achieved with the bipolar electrode arrays of FIG. 4 are electrically activated;

FIGS. 8A-8B are schematic illustrations of the progression of tissue ablation achieved with the bipolar electrode arrays of FIG. 5 are electrically activated;

FIG. 9 is a perspective view of one embodiment of a bipolar electrode array probe constructed in accordance with the present invention, wherein the electrode arrays are particularly shown deployed;

FIG. 10 is a perspective view of the distal end of the probe of FIG. 9;

FIG. 11 is a perspective view of the probe of FIG. 9, wherein the electrode arrays are particularly shown retracted;

FIG. 12 is a perspective view of the distal end of the probe of FIG. 11;

FIG. 13 is a perspective view of the distal end of the probe of FIG. 12, particularly showing a layer of insulation for enhancing the electrical characteristics of the probe;

FIG. 14 is a perspective view of another embodiment of a bipolar electrode array probe constructed in accordance with the present invention, wherein the electrode arrays are particularly shown deployed;

FIG. 15 is a perspective view of the distal end of the probe of FIG. 14;

FIG. 16 is a perspective view of the distal end of the probe of FIG. 15, particularly showing a layer of insulation for enhancing the electrical characteristics of the probe;

FIG. 17 is a perspective view of still another embodiment of a bipolar electrode array probe constructed in accordance with the present invention, wherein the electrode arrays are particularly shown deployed; and

FIG. 18 is a close-up perspective view of the distal electrode array of the probe of FIG. 17, particularly showing a layer of insulation for enhancing the electrical characteristics of the probe.

### DETAILED DESCRIPTION OF THE ILLUSTRATED EMBODIMENTS

Generally, the invention is directed to the use of RF electrode arrays, particularly bipolar electrode arrays, for the ablation of treatment regions within solid tissue of a patient. The treatment regions may be located anywhere in the body where hyperthermic exposure may be beneficial. Most commonly, the treatment region will comprise a solid tumor within an organ of the body, such as the liver, lung, kidney, pancreas, breast, prostate (not accessed via the urethra), uterus, and the like. The volume to be treated will depend on the size of the tumor or other lesion, but embodiments of the invention are particularly suitable for treating relatively large tissue regions. The peripheral dimensions of a particular treatment region may be regular, e.g., spherical or ellipsoidal, but will more usually be somewhat irregular. The lesion created to enclose the target tissue region utilizing embodiments of this invention will usually be cylindrical or a truncated conical volume, as described in more detail below. The treatment region may be identified using conventional imaging techniques capable of elucidating a target tissue, e.g., tumor tissue, such as ultrasonic scanning, magnetic resonance imaging (MRI), computer-assisted tomography (CAT), fluoroscopy, nuclear scanning (using radiolabeled tumor-specific probes), and the like. Preferred is the use of high-resolution ultrasound, which can be employed to monitor the size and location of the tumor or other target tissue, being treated, either intraoperatively or externally.

Apparatus according to embodiments of the invention will usually comprise at least one probe having a distal end adapted to be positioned beneath a tissue surface at or near the treatment region or regions. A first array of electrodes comprising a plurality of tissue-penetrating electrodes, typically in the form of sharpened, small cross-section metal elements are reciprocatably attached to the probe so that they penetrate into tissue as they are advanced from a first specific site (referred to hereinafter as the first target site) at or adjacent to a peripheral boundary of the treatment region, as described in more detail hereinafter. The primary requirement of such electrode elements is that they can be deployed in an array, preferably a three-dimensional array, emanating from the first treatment site within the treatment region of the, tissue. Usually, the first electrode array will be deployed from a first target site on a “distal” side of the treatment region, i.e., the side that is most remote from the organ or tissue entry point. In the exemplary embodiments, the electrode elements are first introduced to the treatment region in a radially collapsed or other constrained configuration, and thereafter advanced into the tissue from a delivery cannula or other element in a divergent pattern to achieve the desired three-dimensional array. The electrode elements will diverge radially outwardly from the delivery cannula (located at the first target site) in a uniform pattern, i.e., with the spacing between adjacent electrodes diverging in a substantially uniform and/or symmetric pattern. Preferably, adjacent electrodes will be spaced-apart from each other in similar or identical, repeated patterns and will usually be symmetrically positioned about an axis of the delivery element. The electrode elements may extend or project along generally straight lines from the probe, but will more usually be shaped to curve radially outwardly and to evert proximally so that they face partially or fully in the proximal direction when fully deployed. It will be appreciated that a wide variety of particular patterns can be provided to uniformly cover the region to be treated.

Apparatus according to embodiments of the invention will also comprise at least a second array of electrodes comprising a plurality of tissue-penetrating electrodes typically in the form of sharpened, small cross-section metal wires or elements. The second electrode array will usually be attached to the same probe as is the first electrode array. In some instances, however, the use of such embodiments may utilize first and second electrode arrays, which are deployed from separate probes and operated in a bipolar manner, as, described in more detail below. The electrode wires or elements of the second array will be deployed from a second target site within the treatment region, usually on a “proximal” side thereof, i.e., the side which is closest to the organ or tissue entry point. The electrodes of the second array will be introduced similarly to those of the first array, i.e., in a collapsed configuration, and subsequently deployed radially outwardly. In the exemplary embodiments, both the first and the second electrode arrays include everting electrode elements, which form arrays having generally concave and convex surfaces. By facing the concave surfaces and electrode tips of the two electrode arrays toward each other so that they are generally aligned along a common axis, usually defined by a shaft of the probe, radiofrequency and other high frequency currents may be applied to tissue in a manner which creates a uniform lesion, i.e., a lesion which is continuous and without significant portions of viable tissue, even when the region has portions which have different perfusion and different cooling characteristics.

Referring now to FIGS. 3-5, a system 6 comprising a first electrode element 8 and a second electrode element 10 is schematically show. The first and second electrode elements 8 and 10 include respective first and second electrode arrays 12 and 14 that are shown as fully everting arrays, where individual electrode wires extend first in an axial direction, diverge radially outwardly, and turn back upon themselves until they face in an opposite direction from which they began. The first electrode element 8 further includes a first axial electrode stem 16, which extends along an axis line 18 toward a second axial electrode stem 20, which is part of the second electrode element 10.

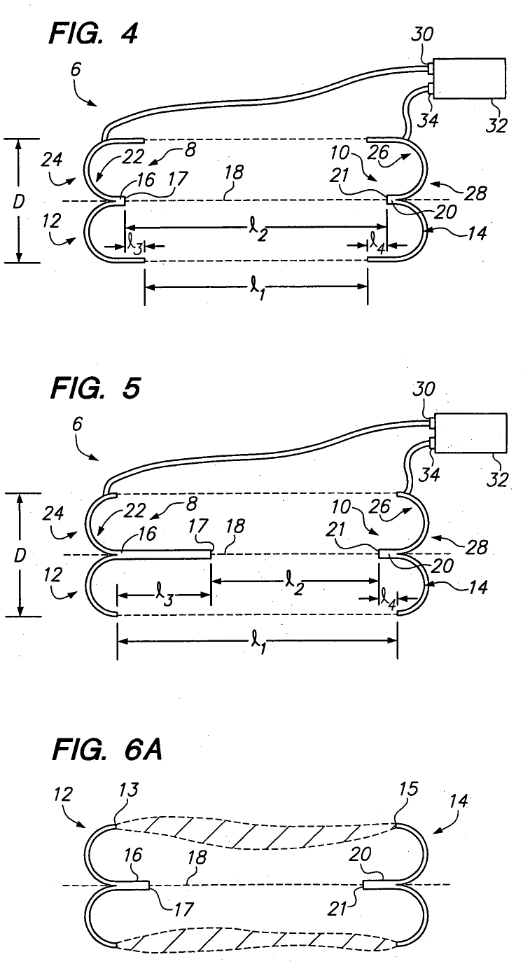

The first electrode array 12 has a concave surface 22 and a convex surface 24, and the second electrode array 14 also has a concave surface 26 and a convex surface 28. In the illustrated embodiment, the concave surfaces at 22 and 26 of the electrode arrays 12 and 14 face each other along the axis line 18, so that a distance l₁ is defined between distal termini 13 and 15 (i.e., the tips of the electrode wires) of the electrode arrays 12 and 14. The axial electrode stems 16 and 20 also face each other and extend toward each other, leaving a distance l₂ between the inner termini 17 and 21 (i.e., the tips) of the electrode stems 16 and 20. FIGS. 3 and 5 show the distance l₂ as being less than the distance l₁, whereas FIG. 4 shows the distance l₂ as being greater than the distance l₁. As will be described in further detail below, the radial shape of an ablation lesion created by the system 6 about the axis 18 can be modified via selection of the distances l₁ and l₂ relative to each other.

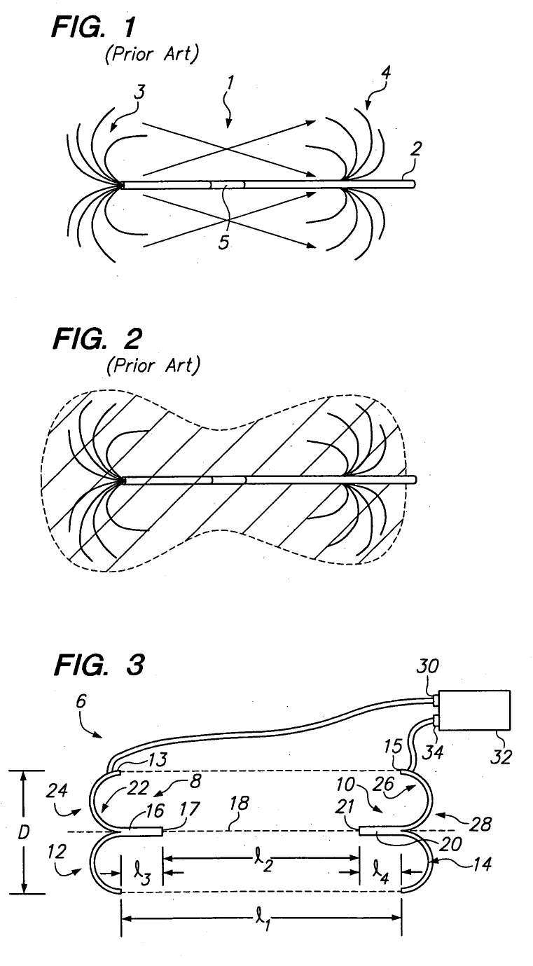

As shown in FIG. 2, due mainly to the greater distance l₁ relative to distance l₂, the inner terminal 17 of the first electrode stem 16 extends axially inwardly beyond the distal terminal 13 of the first electrode array 12, thereby defining a distance l₃ therebetween, and the inner terminal 21 of the second electrode stem 20 extends axially inwardly beyond the distal terminal 15 of the second electrode array 14, thereby defining a distance l₄ therebetween. In contrast, as shown in FIG. 4, due mainly to the greater distance l₂ relative to distance l₁, the distal terminal 13 of the first electrode array 12 extends axially inwardly from the inner terminal 17 of the first electrode stem 16, thereby defining a distance l₃ therebetween, and the distal terminal 15 of the second electrode array 14 extends axially inwardly beyond the inner terminal 21 of the second electrode stem 20, thereby defining a distance l₄ therebetween.

As shown in FIGS. 3 and 4, the distances l₃ and l₄ are equal to each other, so that an ablation lesion created by the system 6 will be somewhat symmetrical along the axis 18, as will be described in further detail below. In contrast, as shown in FIG. 5, the distances l₄ and l₄ are not equal to each other, so that an ablation lesion created by the system 6 will be somewhat asymmetrical along the axis 18, or a symmetrical ablation lesion can be created by the system 6 within a treatment region with asymmetrical tissue properties, as will be described in further detail below.

In exemplary methods of the invention, the electrode arrays 12 and 14 will be disposed within tissue on opposite sides of a treatment region. The arrays will be disposed generally as shown in FIGS. 3-5, preferably with the axial electrode stems 16 and 20 aligned along the axis 18, most preferably being positioned on a single probe shaft, as will be described in more detail hereinafter. The first electrode element 8 is connected to a first pole 30 of a radiofrequency power supply 32. The second electrode element 10 is connected to the other pole 34 of the power supply 32. In this way, the electrode elements 8 and 10 will be powered in bipolar manner in order to effect radiofrequency current flow through the tissue volume between the arrays. Tissue destruction by the current will define the treatment region.

The RF power supply 32 may be a conventional general purpose electrosurgical power supply operating at a frequency in the range from 300 kHz to 1.2 MHz, with a conventional sinusoidal or non-sinusoidal wave form. Such power supplies are available from many commercial suppliers, such as Valleylab, Aspen, and Bovie. Most general purpose electrosurgical power supplies, however, are constant current, variable voltage devices and operate at higher voltages and powers than would normally be necessary or suitable. Thus, such power supplies will usually be operated initially at the lower ends of their voltage and power capabilities, with voltage then being increased as necessary to maintain current flow. More suitable power supplies will be capable of supplying an ablation current at a relatively low fixed voltage, typically below 200 V (peak-to-peak). Such low voltage operation permits use of a power supply that will significantly and passively reduce output in response to impedance changes in the target tissue. The output will usually be from 50 W to 300 W, usually having a sinusoidal wave form, but other wave forms would also be acceptable. Power supplies capable of operating within these ranges are available from commercial vendors, such as Boston Scientific Therapeutics Corporation. Preferred power supplies are model RF-2000 and RF-3000, available from Boston Scientific Corporation.

The geometry and volume of the treatment region within the patient tissue can determined by controlling various dimensions of the apparatus. For example, the arrays 12 and 14 will usually have outer circular diameters D in the range from 1 cm to 6 cm, usually from 2 cm to 4 cm. The diameters of each array will usually be the same, although they could differ in certain circumstances. When the diameters are the same, the geometry of the lesion created will be generally cylindrical. When the diameters are different, the geometry could generally be a truncated cone. The distance X, between the inner termini 13 and 15 of the electrode arrays 12 and 14 will usually be in the range from 2 cm to 10 cm, more usually in the range from 3 cm to 7 cm, and preferably in the range from 4 cm to 6 cm. The axial electrode stems 16 and 20 will typically have a length in the range from 0.0 cm (i.e., non-existent) to 2 cm.

Based on the distance l₁, the desired shape of the resulting ablation lesion, and the desired ablation time, the distance 2 between the inner termini 17 and 21 of the axial stems 16 and 20 is selected to control the proportion of current flowing between the distal termini 13 and 15 of the electrode arrays 12 and 14 relative to the current flowing between the inner termini 17 and 21 of the electrode stems 16 and 20.

It has been discovered that, in general, the greater the proportion of current flowing between the distal termini 13 and 15 of the electrode arrays 12 and 14, as opposed to current flowing between the inner termini 17 and 21 of the electrode stems 16 and 20, the more uniform the resulting ablation lesion will be along the periphery of the tissue treatment region (e.g., so that the ablation lesion is more cylindrical, rather than hour-glass shaped), but the greater the time needed to ablate the tissue core along the axis 18. In contrast, the greater the proportion of current flowing between inner termini 17 and 21 of the electrode stems 16 and 20, the less uniform the resulting ablation lesion will be along the periphery of the tissue treatment region, but the lesser the time needed to ablate the tissue core along the axis 18.

With this phenomenon in mind, the distance l₂ is preferably selected relative to the distance l₁, such that a majority of the current, and preferably substantially all of the electrical current, will essentially flow between distal termini 13 and 15 of the electrode arrays 12 and 14, while a small or minimal amount of current flows between the inner termini 17 and 21 of the electrode stems 16 and 20. It has been discovered that the preferred distance l₂ should be greater than fifty percent of the distance l₁ in order to ensure that the majority of the electrical current flows between the distal termini 13 and 15 of the respective electrode arrays 12 and 14. Optimally, the distance l₂ should be greater than seventy-five percent of the distance l₁ in order to ensure that substantially all of the electrical current flows between the distal termini 13 and 15 of the respective electrode arrays 12 and 14. In some cases, the distance l₂ may be greater than the distance l₁, as illustrated in FIG. 4, although, in other cases, it may be desirable that the distance l₂ be less than the distance l₁, so that some electrical current flows between the electrodes stems 16 and 20 to aid in ablating tissue along the axis 18, thereby reducing the tissue ablation time.

Thus, it will be appreciated that by increasing the proportion of current flowing between the distal termini 13 and 15 of the electrode arrays 12 and 14 and increasing the ablation time, the treatment region will be heated and necrosed from the outer regions inward towards the center region, thus enhancing the ability to completely and uniformly necrose the entire tissue volume of the treatment region defined by the outward perimeters of the arrays 12 and 14.

It has been discovered that the axial symmetry of a resulting ablation lesion can be modified by selecting the distances l₃ and l₄ relative to each other. In particular, as the distance l₄ decreases relative to the distance l₃ (assuming the arrangement in FIGS. 3 and 5) or increases relative to the distance l₃ (assuming the arrangement in FIG. 4), more electrical current will be focused in the peripheral tissue region adjacent the electrode array 14. In contrast, as the distance l₄ increases relative to the distance l₃ (assuming the arrangement in FIGS. 3 and 5) or increases relative to the distance l₃ (assuming the arrangement in FIG. 4), more electrical current will be focused in the peripheral tissue region adjacent the electrode array 12. Thus, it can be appreciated that the electrode array with the higher peripheral concentration of electrical current can be located in a portion of the treatment region requiring an increased ablation power (e.g., if such tissue portion is radially larger than the remaining portion of the treatment region, or if such tissue portion is adjacent a blood vessel that conducts heat away from it).

Referring to FIGS. 6A-6B, the ideal propagation of the tissue necrosis region achieved by the embodiment of FIG. 3 will now be described. Initially, the current flux is concentrated between the distal termini 13 and 15 of the electrode arrays 12 and 14, resulting in a generally hollow cylindrical necrosis region at the periphery of the treatment region, as shown in FIG. 6A. As the tissue become necrosed, its impedance increases, causing the current flux to move inwardly between the inner termini 17 and 21 of the electrodes stems 16 and 20 to ablate the tissue core along the axis 18, resulting in a solid cylindrical necrosis region, as shown in FIG. 6B. Usually, the region of necrosis will extend slightly beyond the arrays themselves due to heat conduction from the tissue, which is being directly heated by the electrical current flow. In addition to the impedance increase, the reduction of blood flow through the central portions of the treatment region as that tissue becomes necrosed will also contribute to the uniformity of heating and subsequent necrosis of the larger volume. That is, as the blood flow through the treatment region is decreased, the ability to uniformly heat the tissue via the passage of current is enhanced.

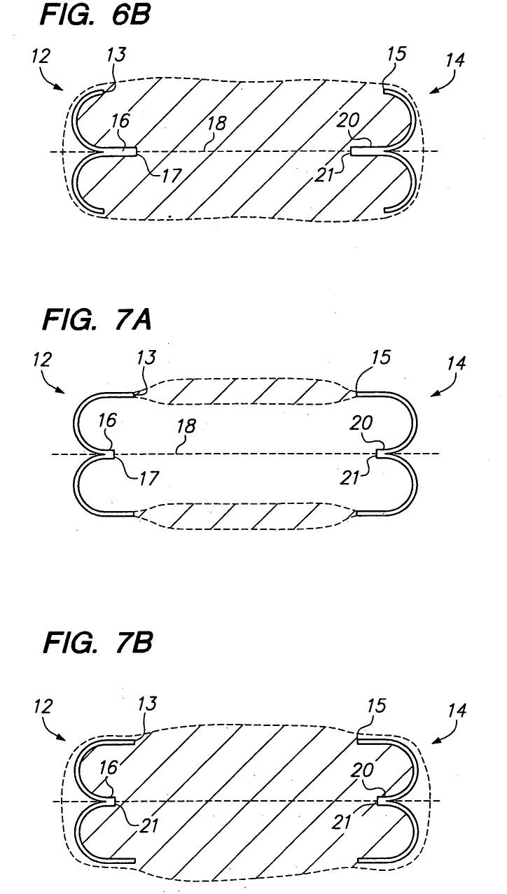

Referring to FIGS. 7A-7B, the ideal propagation of the tissue necrosis region achieved by the embodiment of FIG. 4 will now be described. As with the embodiment of FIG. 3, the current flux will be concentrated between the distal termini 13 and 15 of the electrode arrays 12 and 14, resulting in a generally hollow cylindrical necrosis region at the periphery of the treatment region, as shown in FIG. 7A. In this case, however, because a small or minimal amount of current will flow between the inner termini 17 and 21 of the electrode stems 16 and 20, the necrosis region is uniform along the axis 18. Preferably, because the electrode arrays 12 and 14 are solely used to ablate the core tissue region, the magnitude of the current is reduced, so that the tissue impedance does not rise too sharply. As a result, given enough time, the tissue core along the axis 18 will necrose via heat conduction, thereby resulting in a solid cylindrical necrosis region, as shown in FIG. 7B.

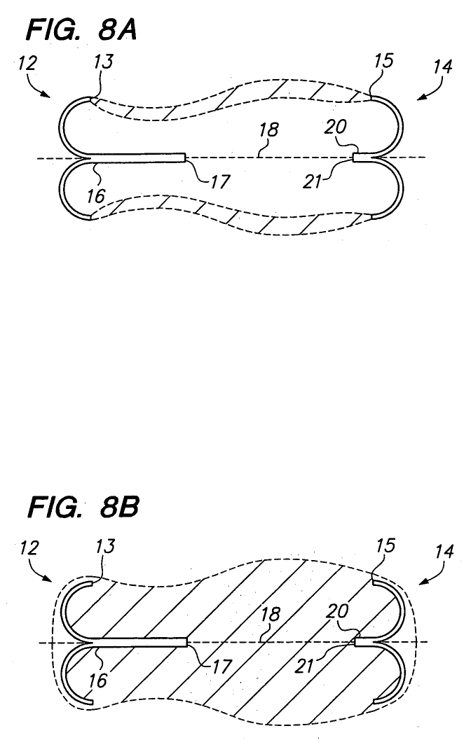

Referring to FIGS. 8A-8B, the ideal propagation of the tissue necrosis region achieved by the embodiment of FIG. 5 will now be described. As with the embodiments of FIG. 3, the current flux will be concentrated between the distal termini 13 and 15 of the electrode arrays 12 and 14. However, the current flux will be more concentrated at the peripheral of the treatment region nearest the electrode array 14. If the treatment region has uniform tissue characteristics, a generally hollow vase-shaped necrosis region will result, as shown in FIG. 8A. If the tissue adjacent the electrode array 12 has either an increased impedance or heat loss, a generally hollow cylindrical necrosis region may result, as previously shown in FIGS. 6A and 7A. As the tissue become necrosed, its impedance increases, causing the current flux to move inwardly between the inner termini 17 and 21 of the electrodes stems 16 and 20 to ablate the tissue core along the axis 18, resulting in a solid vase-like necrosis region, as shown in FIG. 8B, or in the case of a tissue region with asymmetrical tissue characteristics, a solid cylindrical necrosis region, as previously shown in FIGS. 6B and 7B.

The previously described electrode elements 8 and 10 will typically be integrated within a probe for deployment within a patient's body. The probe will usually comprise an elongate shaft, typically a rigid or semi-rigid, metal or plastic cannula. In some cases, the cannula will have a sharpened tip, e.g., be in the form of a needle, to facilitate introduction to the tissue treatment region. In such cases, it is desirable that the cannula or needle be sufficiently rigid, i.e., have sufficient column strength, so that it can be accurately advanced through tissue. In other cases, the cannula may be introduced using an internal stylet, which is subsequently exchanged for one or more of the electrode arrays. In the latter case, the cannula can be relatively flexible since the initial column strength will be provided by the stylet. The cannula serves to constrain the individual electrode elements of the electrode arrays in a radially collapsed configuration to facilitate their introduction to the tissue treatment region. The first electrode array can then be deployed to its desired configuration, usually a three-dimensional configuration, by extending distal ends of the electrode elements from the distal end of the cannula into the tissue. In the preferred case of the tubular cannula, this can be accomplished simply by advancing the distal ends of the electrode elements of the first electrode array distally from the tube so that they emerge and deflect (usually as a result of their own spring or shape memory) in a radially outward pattern. The electrode arrays of the second electrode array may then be proximally advanced from the tube so that they emerge and deflect (again, usually as a result of their own spring or shape memory) in a radially outward pattern, which is a mirror image of the pattern formed by the first electrode array. Particular devices employing a single probe or elongate member for deploying such spaced-apart arrays will be described in more detail below.

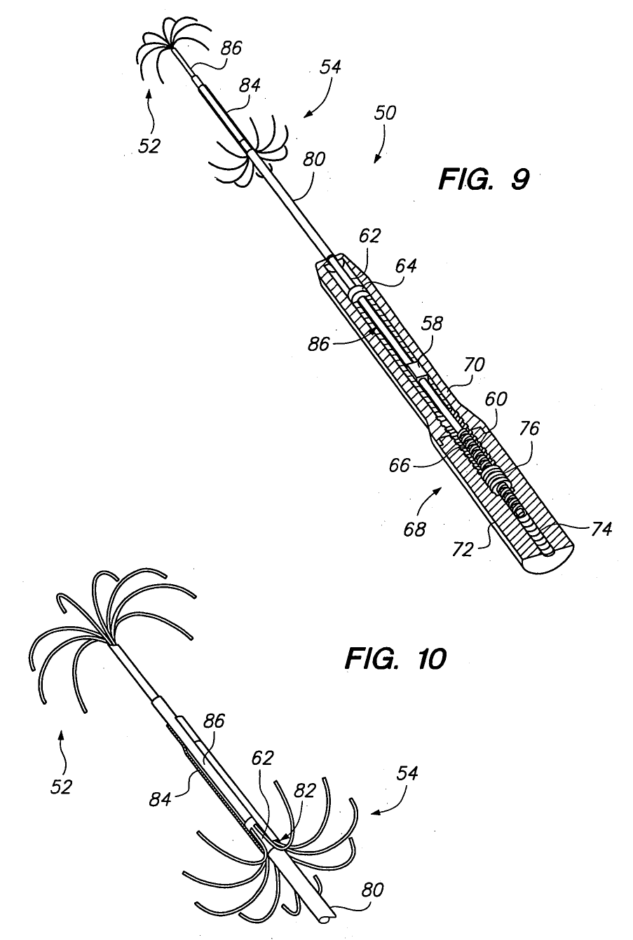

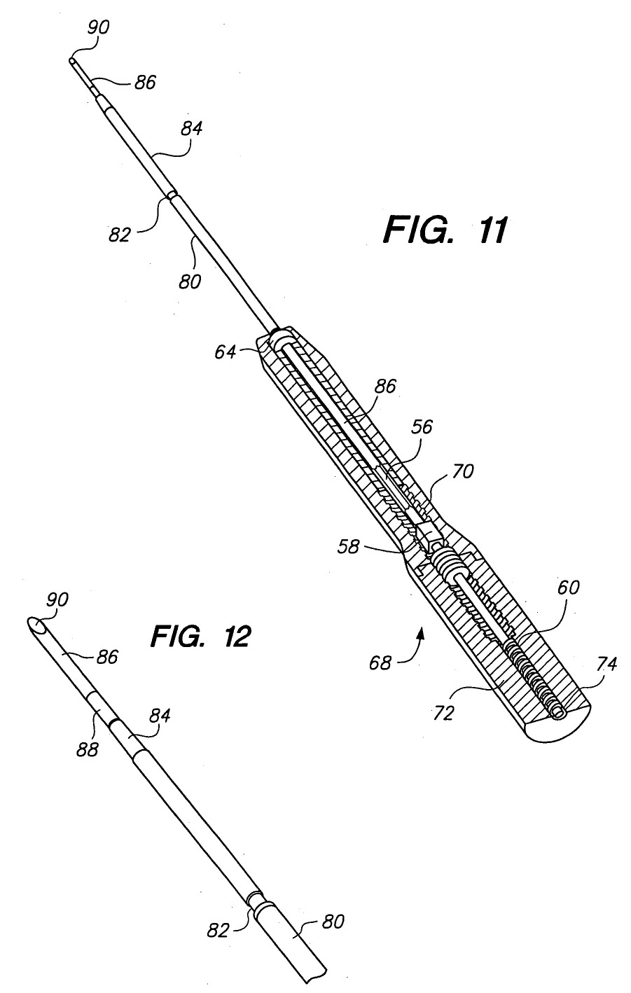

Referring now to FIGS. 9-13, one exemplary electrode probe 50 will be described. The probe 50 has a coaxial design with a distal electrode array 52 and a proximal electrode array 54. The proximal electrode array 54 is attached to a proximal conductor 62 (shown in FIGS. 9 and 10), which in turn, is attached to a proximal yoke 64. The proximal yoke 64 also has a threaded end 66 in a handle 68 of the probe 50. The distal electrode array 52 is deployed by a distal conductor 56 (shown in FIG. 11), which is attached to a slider 58 having a threaded end 60. The distal conductor 56 axially extends within a distal tube 86 mounted within the handle 68. The distal tube 86, with the distal conductor 56, axially extends through the proximal conductor 62 to the distal tip of the probe. To prevent shorting, the portion of the distal tube 86 that extends through the proximal conductor 62 is electrically insulated.

The handle 68, in turn, includes a stationary portion 70 and a rotatable portion 72. The rotatable portion 72 has a first threaded channel 74, which receives the threaded end 60 of the distal array slider 58. A second threaded channel 76 receives the threaded end 66 of the proximal yoke 64. In this way, rotation of the rotatable part 72 of handle 68 will simultaneously advance the distal slider 58 to deploy the distal electrode array 52 and retract the proximal yoke 64 which will deploy the proximal array 54, as best illustrated in FIG. 3.

The proximal conductor 62 extends distally through an insulated outer sheath 80 and past a gap 82 (FIG. 10) between the sheath 80 and a proximal conductive tube 84. When the proximal array 54 is distally advanced, as shown in FIGS. 11 and 12, the proximal array 54 will be contained within a central lumen of the proximal conductive tube 84. As the array 54 is proximally advanced, by rotation of handle portion 72, the individual electrode tines advance radially outwardly through the gap 82 and eventually extend to their fully everted configuration, as shown in FIGS. 9 and 10.

While the proximal array 54 is being proximally deployed, the distal array 52 is simultaneously being deployed by advancing distally outwardly from the distal conductive tube 86 at the distal end of the probe 50. When fully deployed, as shown in FIGS. 9 and 10, the distal electrode array 52 is in electrical contact with the distal conductive tube 86, and the proximal electrode array 54 is in electrical contact with the proximal conductive tube 84. The proximal portion of the distal conductive tube 86, and in particular, the portion extending from the handle 72 to just distal to the proximal conductive tube 84 is electrically insulated, thereby forming a non-conductive gap 88 between the conductive tubes 86 and 84. Alternatively, the proximal and distal arrays 52 and 54 can be separately deployed using deployment mechanisms described in U.S. patent application Ser. No. 09/663,048, which has previously been incorporated herein by reference.

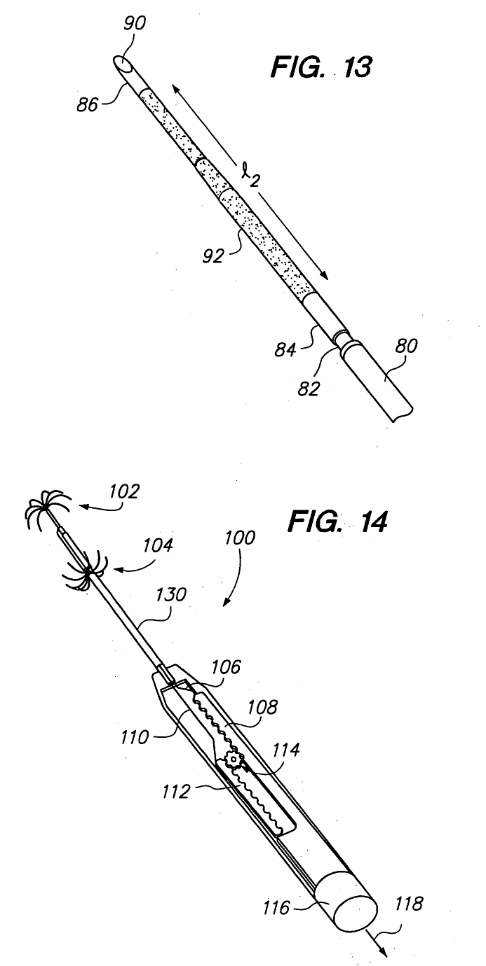

It can be appreciated that the proximal conductive tube 84 effectively forms a proximal electrode stem, such as the stem 20 illustrated in FIGS. 3-5, and the distal conductive tube 86 effectively forms a distal electrode stem similar to the stem 16 illustrated in FIGS. 3-5. As shown in FIG. 13, a layer of electrically insulative material 92 is applied over the proximal conductive tube 84, distal conductive tube 86, and the non-conductive gap 88 in order to shorten the effective electrode stems, thus increasing the distance W2 between the stems. The insulative material 92 may, e.g., be plastic, such as fluorinated ethylene propylene (FEP), rubber, a polymer coating or any other medical grade material suitable for insulating against radio frequency energy.

The insulative material 92 may be applied to the probe 50 equidistantly between the electrode arrays 52 and 54 to form the electrode configuration illustrated in FIGS. 3 and 4 (equal distances l₃ and l₄), or can be shifted towards one of the electrode arrays 52 and 54 to form the electrode configuration illustrated in FIG. 5 (non-equal distances l₃ and l₄). Although shown as partially covering the conductive tubes 84 and 86, the insulative material 92 can cover all of the conductive tubes 84 and 86 to minimize or eliminate the stems. Although the application of a separate insulative material 92 provides a convenient means for shaping the resulting tissue ablation lesion given an existing bipolar ablation probe, it should be noted that the distances l₂, l₃, and l₄ can be selected by adjusting the lengths of the conductive tubes 84 and 84 or by forming portions of the conductive tubes 84 and 86 out of an electrically insulative material. It should also be noted that the insulative material 92 is shown as continuously extending along the probe 50. Alternatively, there may be cylindrical gaps within the insulative material 92. For example, the insulative material 92 may not cover the non-conductive gap 88.

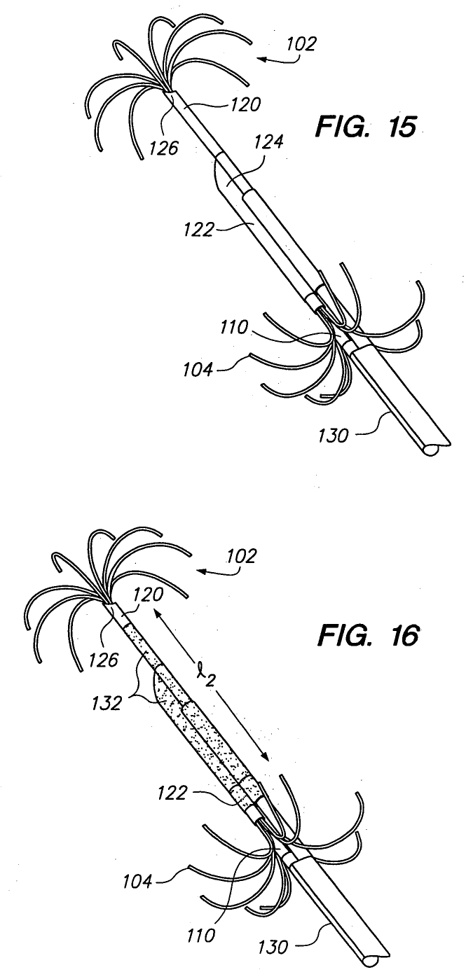

Referring now to FIGS. 14 and 15, a second exemplary probe 100 constructed in accordance with another embodiment of the invention will be described. The probe 100 includes a distal array 102 and a proximal array 104, each of which comprise a plurality of individual everting electrodes which may be similar in construction to those described in connection with probe 50. The distal array 102 is connected through a conductor 106 to a first rack 108. The proximal array 104 is connected to a conductor 110, which is connected to a second rack 112. A pinion gear 114 couples the racks 108 and 112 so that pulling on a knob 116 in a proximal direction (arrow 118) causes the first rack 108 to move proximally and the second rack 112 to move distally. This way, the distal array 102, which is connected to rack 108, will be retracted proximally within the probe while the proximal array 104 will be retracted distally within the probe.

Unlike probe 50, however, the distal array 102 and proximal array 104 are disposed in different, parallel tubular structures. As best shown in FIG. 15, the distal array 102 is disposed in a distal conductive tube 120, and the proximal array 104 is disposed in a proximal conductive tube 122. The distal conductive tube 120 and proximal conductive tube 122 are both electrically conductive so they act as axial conductors in conjunction with their respective arrays. Moreover, an insulated gap 124 exists between the electrically conductive tubes 120 and 122 to provide a gap between them, as generally described previously. Additionally, the distal tip 126 of at least the distal conductive tube 120 will be sharpened to facilitate tissue insertion. Optionally, the insulated gap region 124 at the distal end of the proximal conductive tube 122 may also be tapered or beveled in order to facilitate insertion. As best seen in FIG. 15, the proximal conductor 110 and distal conductor 106 will both extend through parallel tubes, which are covered by an insulating material 130. Thus, the probe 100 may be used in generally the same manner as described for prior probe 50.

It can be appreciated that the proximal conductive tube 122 effectively forms a proximal electrode stem, such as the stem 20 illustrated in FIGS. 3-5, and the distal conductive tube 120 effectively forms a distal electrode stem similar to the stem 16 illustrated in FIGS. 3-5. As shown in FIG. 16, a layer of electrically insulative material 132 is applied over the proximal conductive tube 122, distal conductive tube 120, and the non-conductive gap 124 in order to shorten the electrode stems, thus increasing the distance 2 between the stems in the same manner previously described with respect to the probe 50. The insulative material 132 cover only portions of the conductive tubes 120 and 122, as illustrated in FIG. 16, can alternatively cover all of the conductive tubes 120. The insulative material 132 can equidistantly separate the electrode arrays 102 and 104, as illustrated in FIG. 16, so that the distances l₃ and l₄ are equal, or can be shifted towards one of the electrode arrays 102 and 104 so that the distances l₃ and l₄ are unequal. The insulative material 132 may be continuous, as illustrated in FIG. 16, or can have gaps.

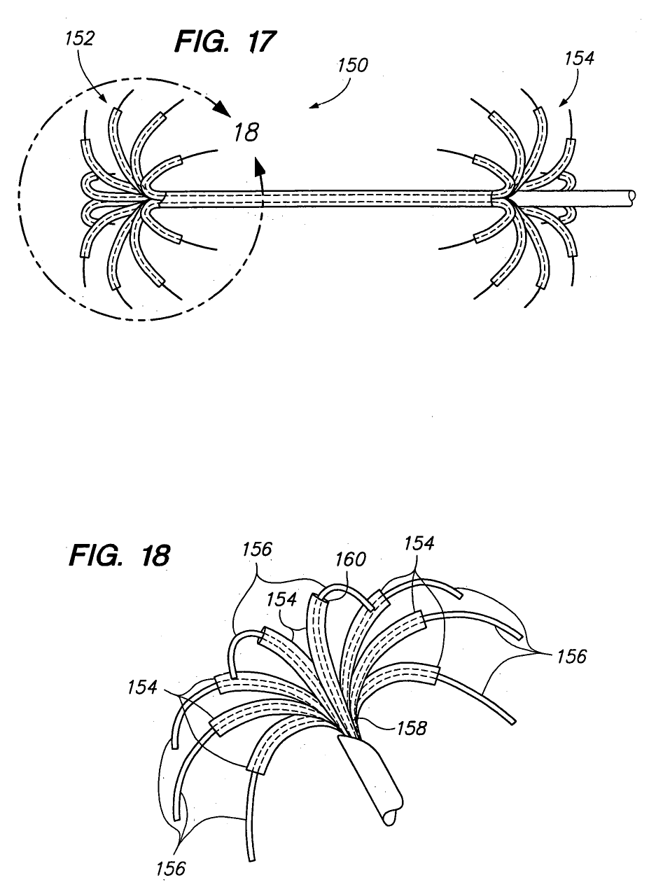

In addition to the provision of insulation on the shafts of the previously described probes 50 and 100, electrical insulation can be also be provided on the needles of the electrode arrays. For example, FIGS. 17 and 18 illustrate a probe 150 with partially insulated electrode arrays 150 and 152. In particular, a length of each needle is partially coated with an insulating material 154, leaving exposed distal needle portions 156. The insulating material may be plastic, such as fluorinated ethylene propylene (FEP), rubber, a polymer coating or any other medical grade material suitable for insulating against radio frequency energy.

As shown, the needles are substantially insulated from a point of deployment 158 to a peak of an arch 160 formed by each needle. In this manner, the electrical current conveyed between the electrode arrays 150 and 152 will be concentrated at the tips 156, thereby maximizing the radius of the resulting ablation lesion. Nevertheless, in some instances, the maximum may not be preferred and therefore, one of skill in the art will appreciate that the needles may be insulated along any portion between the point of deployment 158 and any designated point along the needle. Although all of the needles are shown to be partially insulated, less than all of the needles may be insulated, depending on the desired shaped of the ablation.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

1. A tissue ablation probe, comprising:
a proximal electrode array having a retracted configuration and a deployed configuration;
a distal electrode array having a retracted configuration and a deployed configuration, wherein proximal and distal electrode arrays have distal termini that are separated from each other by a first length when deployed; and
a shaft carrying the proximal and distal electrode arrays, wherein the shaft has an electrically insulative portion that separates the proximal and distal electrode arrays, the insulative shaft portion spanning a second length greater than seventy-five percent of the first length.

2. The probe of claim 1, wherein the electrically insulative portion of the shaft is continuous.

3. The probe of claim 1, wherein the shaft comprises a proximal conductive tube from which the proximal electrode array is deployed, and a distal conductive tube from which the distal electrode array is deployed.

4. The probe of claim 1, wherein the insulative shaft portion comprises an electrically conductive wall and an electrically insulative material disposed on the exterior of the conductive wall.

5. The probe of claim 1, wherein the proximal and distal electrode arrays have respective concave faces that oppose each other when in the deployed configuration.

6. The probe of claim 1, wherein the proximal and distal electrode arrays each comprises a plurality of individual electrodes that initially move axially and then evert as they are deployed.

7. The probe of claim 1, wherein the proximal and distal electrode arrays are electrically isolated from each other.

8. The probe of claim 1, wherein the second length is equal to or greater than the first length.

9. A tissue ablation probe, comprising:
a proximal electrode array having a retracted configuration and a deployed configuration;
a distal electrode array having a retracted configuration and a deployed configuration;
a shaft carrying the proximal and distal electrode arrays, the shaft having an intervening portion between the proximal and distal electrode arrays, the intervening portion having an electrically conductive proximal region, an electrically conductive distal region, and a non-conductive gap therebetween; and
a separate electrically insulative material covering at least portions of the proximal and distal shaft regions.

10. The probe of claim 9, wherein the insulative material covers the non-conductive gap.

11. The probe of claim 9, wherein the shaft comprises a proximal conductive tube from which the proximal electrode array is deployed, and a distal conductive tube from which the distal electrode array is deployed.

12. The probe of claim 9, wherein the proximal and distal electrode arrays have respective concave faces that oppose each other when in the deployed configuration.

13. The probe of claim 9, wherein the proximal and distal electrode arrays each comprises a plurality of individual electrodes that initially move axially and then evert as they are deployed.

14. The probe of claim 9, wherein the proximal and distal electrode arrays are electrically isolated from each other.

15. The probe of claim 9, wherein the insulative material covers the entirety of the intervening shaft portion.

16. The probe of claim 9, wherein the proximal and distal electrode arrays have distal termini that are separated from each other by a first length when deployed, and the insulative material spans a second length greater than fifty percent of the first length.

17. The probe of claim 9, wherein the proximal and distal electrode arrays have distal termini that are separated from each other by a first length when deployed, and the insulative material spans a second length greater than seventy-five percent of the first length.

18. The probe of claim 9, wherein the proximal and distal electrode arrays have distal termini that are separated from each other by a first length when deployed, and the insulative material spans a second length equal to or greater than the first length.

19. The probe of claim 9, wherein the insulative material is closer to one of the proximal and distal electrode arrays than the other.

20. A tissue ablation probe, comprising:
a proximal electrically conductive tube;
a distal electrically conductive tube, wherein the proximal and distal tubes are electrically isolated from each other;
a proximal electrode array proximally deployable from and electrically coupled to the proximal tube;
a distal electrode array distally deployable from and electrically coupled to the distal tube; and
an electrically insulative material covering at least portions of the proximal and distal tubes.

21. The probe of claim 20, wherein the insulative material continuously extends from the proximal tube to the distal tube.

22. The probe of claim 20, wherein the proximal and distal electrode arrays have respective concave faces that oppose each other when in the deployed configuration.

23. The probe of claim 20, wherein the proximal and distal electrode arrays each comprises a plurality of individual electrodes that initially move axially and then evert as they are deployed.

24. The probe of claim 20, wherein the proximal and distal electrode arrays are electrically isolated from each other.

25. The probe of claim 20, wherein the insulative material covers the entirety of the proximal and distal tubes.

26. The probe of claim 20, wherein the proximal and distal electrode arrays have distal termini that are separated from each other by a first length when deployed, and the insulative material spans a second length greater than fifty percent of the first length.

27. The probe of claim 20, wherein the proximal and distal electrode arrays have distal termini that are separated from each other by a first length when deployed, and the insulative material spans a second length greater than seventy-five percent of the first length.

28. The probe of claim 20, wherein the proximal and distal electrode arrays have distal termini that are separated from each other by a first length when deployed, and the insulative material spans a second length equal to or greater than the first length.

29. A tissue ablation probe, comprising:
a proximal electrode element including a proximal electrode stem and a deployable proximal electrode array having distal termini and being electrically coupled to a proximal end of the proximal electrode stem when deployed;
a distal electrode element including a distal electrode stem and a deployable distal electrode array having distal termini and being electrically coupled to a distal end of the distal electrode stem when deployed;
wherein a majority of electrical energy conveyed between the proximal and distal electrode elements is conveyed between distal termini of the proximal and distal electrode arrays.

30. The probe of claim 29, wherein substantially all of the electrical energy conveyed between the proximal and distal electrode elements is conveyed between distal termini of the proximal and distal electrode arrays.

31. The probe of claim 29, wherein the proximal electrode stem comprises a proximal conductive tube from which the proximal electrode array is deployed, and the distal electrode stem comprises a distal conductive tube from which the distal electrode array is deployed.

32. The probe of claim 29, wherein the proximal and distal electrode arrays have respective concave faces that oppose each other when deployed.

33. The probe of claim 29, wherein the proximal and distal electrode arrays each comprises a plurality of individual electrodes that initially move axially and then evert as they are deployed.

34-55. (canceled)
